(19) United States
(12) Patent Application Publication
(10) Pub. No.: US 2006/0183168 A1
(43) Pub. Date: Aug. 17, 2006

(54) Method for optimizing therapeutic efficacy of nemorubicin

(76) Inventors: Maria Cristina Geroni, MILAN (IT); Maria Adele Pacciarini, Milan (IT)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/533,017

(22) PCT Filed: Oct. 8, 2003
(86) PCT No.: PCT/EP03/11180

§ 371 Date: Dec. 12, 2005

(30) Foreign Application Priority Data
Oct. 28, 2002 (EP) 02079489.7

(51) Int. Cl. G01N 33/574; A61K 31/704

(52) U.S. Cl. 435/7.23; 514/34

(57) ABSTRACT

The present invention relates to products and methods for characterizing cancer patients in order to predict therapeutic benefit with a drug metabolized by CYP3A, especially nemorubicin.

### FIELD OF THE INVENTION

The present invention pertains to a new approach to anticancer treatments. More specifically, the invention relates to methods for determining a patient individualized dosage of a drug primarily metabolized by cytocrome P4503A isoenzyme, especially nemorubicin, in order to decrease toxicity and increase efficacy of the chemotherapeutic treatments.

### DESCRIPTION OF THE INVENTION

The present invention relates to the field of cancer treatment and, more particularly, it relates to a method for optimizing the treatment of cancer with chemotherapy by measuring cytocrome P4503A isoenzyme (CYP3A) levels in patients undergoing chemotherapeutic treatment.

It has recently been found that nemorubicin is metabolized primarily by CYP3A4.

The cytocrome P450 (CYP) enzymes constitute a large superfamily of haem-containing proteins that play a central role in the metabolism of a wide variety of endogenous compounds and foreign chemicals, including drugs (Nelson et al, Pharmacogenetics, 1996). In mammals, the main drug metabolizing families of CYP (CYP1, CYP2, CYP3) are primarily expressed in the liver, although specific isoforms are present in some extrahepatic tissues (de Waziers et al, J. Pharmacol. Exp. Ther. 1990). CYP3A4, the most abundantly expressed CYP enzyme in adult human liver, may account for the oxidative metabolism of more than 60% of all clinically used drugs, including anticancer agents such as cyclophosphamide, ifosfamide, paclitaxel, vinblastine and epipodophyllotoxins (Chang et al Cancer Res. 1993; Kivisto et al., Br. J. Clin Pharmacol. 1995; Shimada et al., J. Pharmacol. Exp. Ther. 1994).

It was reported that there was a great interindividual variation in the CYP expression (Shimada et al., J. Pharmacol. Exp. Ther. 1994).

Moreover, CYP3A enzymes are expressed at different levels in human tumors (de Waziers et al, J. Pharmacol. Exp. Ther. 1990; Murray et al., J. Pathol. 1995), and can be inhibited or induced by a number of drugs (Waxman, Arch Biochem. Biophys. 1999). Thus, the expression of CYP3A may profoundly affect the activity and/or the host toxicity of antitumor agents, which are substrates of these enzymes. Moreover, clinically applicable techniques capable of predicting CYP3A4-levels in humans are available (Rivory et al. Clin. Cancer Res. 2000).

Nemorubicin is a doxorubicin derivative currently undergoing clinical evaluation. Previous studies suggest that nemorubicin undergoes hepatic biotransformation into a more cytotoxic metabolite(s). These metabolites have been identified and their antitumor activity and toxicity have been tested (Geroni et al., Proc. Am. Assoc. Cancer Res., 1997). In experimental tumor models, all tested metabolites of nemorubicin resulted as active as the parent compound. As regards potency, one of the identified metabolites presented higher potency in respect to nemorubicin, being its maximum tolerated dose more than five times lower than that of the parent compound.

More recently, the metabolic pathway of nemorubicin has been investigated.

The following EXPERIMENTAL PART illustrates, for example, the role of CYP3A in the metabolic pathway of nemorubicin.

Experimental Part

Materials and Methods

Antibody Studies

Human liver microsomes were preincubated at 25° C. for 5 min. with and without a monoclonal antibody anti-CYP3A4/5 (MAB-3A4 Genetest) in Tris 0.3M (pH 7.4) before adding nemorubicin (20 μM and NADPH (0.5 mM). After 10 min at 37° C., the amount of nemorubicin metabolites was evaluated by a HPLC system.

Metabolic Potential of Microsomes Obtained from Cells Expressing Single Human CYP 450 Isoenzymes

Microsomes were obtained from cell cultures overexpressing CYP3A4, CYP3A5, CYP1A2, CYP2E1, CYP2D61, CYP2C91 and CYP2C8. Microsomes (50 pmol CYP/ml) were incubated with nemomubicin (20 μM) and NADPH (0.5 mM) in 0.3M Tris (pH 7.4) at 37° C. for 20 min. Nemorubicin metabolism was quantified by HPLC method.

HPLC Analysis

High-performance liqid chromatographic (HPLC) system consisted of a Waters Model 510 isocratic pump equipped with autosampler. Detection was performed by a Perkin Elmer fluorescence spectrofotometer LS-5 set at 479 and 552 nm excitation and emission wavelength, respectively. Detector was connected to a Shimadzu C-R3A integrator. The cromatographic separation was performed on a Waters Simmetry C8 reverse phase column. The mobile phase was 10 mM KH₂P₄/Methanol/CH₃CN (45:30:25). The flow rate was 1.5 ml/min. Standard curves of fluorescence versus drug concentration for nemorubicin and metabolites were used to calculate drug concentration in the samples.

Correlation Between CYP3A Expression and Nemorubicin Metabolism in Human Microsomes from Different Patients

Nemorubicin was incubated with liver microsomes from 9 different patients. All human liver microsome samples were tested for the expression of CYP3A by erythromycin-demethylase test (Watkins et al. J. Clin. Invest. 1993). The amount of nemorubicin metabolites was evaluated by a HPLC system and correlated to the expression of CYP3A.

Results

The metabolism of nemorubicin correlates with the levels of CYP3A in human liver samples. More specifically there was a strict correlation only with the expression of CYP3A enzymatic activity (r² 0.993) and not with other CYP isoenzymes such as CYP1A2 (r² 0.0014), CYP2D6 (r² 0.0047), CYP2C9 (r² 0.45) and CYP2C19 (r² 0.0032). The inhibition of nemorubicin metabolism by human liver microsomes was tested using antibodies raised against specific cytocrome P-450 isoenzymes. Obtained results showed that only the CYP3A4 isoenzyme is responsible for the metabolism of nemorubicin. This finding was further supported by the studies performed with microsomes obtained from cells tranfected for the overexpression of different CYP isoenzymes. Only microsomes from CYP3A4-overexpressing cells were able to metabolize nemorubicin. The evidence from the above experiments indicates that the CYP3A4 family of cytocrome P450s is involved in the metabolism of nemorubicin.

The above-obtained results indicate a major role of CYP3A-mediated drug metabolism in the transformation of nemorubicin. Consequently, being the antitumor activity and host toxicity of nemorubicin affected by the level of active/cytotoxic metabolite/s, the CYP3A expression could play a fundamental role in the pharmacological profile of this drug.

Nemorubicin may be therefore considered as an example of an excellent candidate for an individualized therapy because it is metabolized primarily by CYP3A, an enzyme that is known to have interindividual variability.

There is therefore a need to identify levels of CYP3A in a patient in need of a treatment with a drug, which is metabolized primarily by CYP3A, so that administration of said drug can be optimized in view of CYP3A enzymatic profile. Particularly, there is a need to identify levels of CYP3A in a patient in need of nemorubicin treatment, so that nemorubicin administration can be optimized in view of CYP3A enzymatic profile.

The present invention fulfills such a need by providing a method for treating a patient in need of a treatment with a drug which is metabolized primarily by CYP3A, especially nemorubicin, which comprises detecting CYP3A levels in said patient.

In particular, the present invention is directed to a method for optimizing therapeutic efficacy of a drug which is metabolized primarily by CYP3A, especially nemorubicin, in a patient in need thereof, which comprises predicting the sensitivity of a patient towards said drug through the detection of CYP3A levels in a biological sample of said patient and selecting a therapeutically effective amount of said drug based on the above CYP3A levels.

A further object of the present invention is a method for treating a cancer sensitive to a drug which is metabolized primarily by CYP3A, especially nemorubicin, which comprises:

(a) obtaining a biological sample from a patient suffering from said cancer,

(b) detecting the amount of cytochrome CYP3A in said sample; and

(c) selecting a therapeutically effective amount of said drug, based on the above cytochrome CYP3A levels.

Another object of the present invention is a method for predicting patient's sensitivity to a drug, wherein said drug is metabolized by CYP3A, especially nemorubicin, said method comprising determining levels of CYP3A in said patient and wherein the patient's sensitivity to said drug is effected by CYP3A activity.

A kit for detecting the amount of CYP3A in a biological sample for use in a method for treating a cancer sensitive to a drug primarily metabolized by CYP3A, especially nemorubicin, as described in the present specification is also within the scope of the present invention.

For example, according to the patient specific optimal dosing regimen embodiment of the present invention, patients who are candidates for therapy with a drug primarily metabolized by CYP3A (e.g. nemorubicin) provide, e.g. a biological fluid sample for analysis prior to initiation of the treatment. Suitable, rapid and noninvasive methods and kits such as, e.g., erytromycin breath test EBT (Rivory et al, Clin Cancer Res. 2000), are commercially available for testing CYP3A expression in patients.

EBT is a putative in vivo probe for drug metabolism by cytocrome P4503A. As an example, specimens of blood may be collected for testing EBT as a tool for predicting metabolism of a drug primarily metabolized by CYP3A, e.g. nemorubicin (Rivory et al. Clin. Cancer Res. 2000). Since specific cytochrome P4503A levels determine the tolerance of individual patients to a particular dose of the above mentioned drug, a math formula can be applied to calculate a starting dose that minimizes an individual patient's risk of toxicity and maximizes a patient's probability of a therapeutic responses on the basis of levels of CYP3A found in the biological samples collected from the patient under examination. Such an individually adapting starting dose can be greater or smaller than the starting doses determined empirically in clinical trials that did not take into account the enzymatic profile of clinical trial participants.

As used herein, “detection” refers to CYP3A level determination in patients to be treated with nemorubicin.

As used herein, “anticancer therapy” refers to all types of therapies for treating cancers or neoplasms or malignant tumors found in mammals comprising humans, including leukemiae, melanoma, liver, breast, ovary, prostate, stomach, pancreas, lung, kidney, colon and central nervous system tumors.

The phrase “therapeutically effective amount” is intended to qualify the amount of nemorubicin, which should be administered to patients, based on the CYP3A level.

As already said, nemorubicin may be used in anticancer therapy for treating, e.g. breast, ovary, prostate, lung, colon, kidney, stomach, pancreas, liver, melanoma, leukemiae and central nervous system tumors in mammals, including humans. In a preferred embodiment, nemorubicin may be useful for treating a liver cancer, for example a liver cancer primarily confined to the liver such as, e.g. a hepatocellular carcinoma or a cholangiocarcinoma, or liver metastases.

Nemorubicin can be administered to a patient in any acceptable manner that is medically acceptable including orally, parenterally, or with locoregional therapeutic approaches such as, e.g., implants. Oral administration includes administering nemorubicin in a suitable oral form such as, e.g., tablets, capsules, lozenges, suspensions, solutions, emulsions, powders, syrups and the like. Parenteral administration includes administering nemorubicin by subcutaneous, intravenous or intramuscular injections. Implants include intra artherial implants, for example an intrahepatic arthery implant.

Injections and implants are preferred administration routes for nemorubicin because they permit precise control of the timing and dosage levels used for administration.

For example, for treating a patient suffering from a liver cancer as defined above, intrahepatic administration of nemorubicin may be performed via the hepatic artery. More precisely, nemorubicin may be administered to a patient with either a hepatic metastatic cancer, or with previously untreated primary liver carcinoma, via the hepatic artery directly into the lateral entry of an i.v. line inserted into the bung of an intrahepatic potacath or via a catheter inserted into the hepatic artery.

The actual preferred method of administration of nemorubicin may vary according to, inter alia, the particular cancer being treated, the severity of the disease state being treated, and the particular patient being treated.

Pharmaceutically acceptable carriers or excipients to be utilized in the preparation of a pharmaceutical composition comprising nemorubicin as an active ingredient are well known to people skilled in the art of formulating compounds in a form of pharmaceutical compositions.

For example, such pharmaceutical compositions may routinely contain, e.g., pharmaceutically acceptable salts, buffering agents, preservatives and/or compatible carriers. As used-herein, “pharmaceutically acceptable carrier” refers to one or more compatible solid or liquid filler, diluent or encapsulating substances which are suitable for administration to mammals including humans.

Pharmaceutical compositions suitable for parenteral or intrahepatic administration are formulated in a sterile form.

The sterile composition thus may be a sterile solution or suspension in a non-toxic parenterally acceptable diluent or solvent.

Pharmaceutical compositions for intrahepatic administration are formulated, for example, in a form, which remains selectively in a liver tumor after their injection through the hepatic artery; LIPIODOL™ is a suitable carrier of anticancer agents, which can be used for intrahepatic administration.

The amount of an active ingredient contained in the pharmaceutical composition according to the invention may vary quite widely depending upon many factors such as e.g. the administration route and the vehicle.

As an example, the pharmaceutical composition of the invention may contain from 0.1 mg to 100 mg of nemorubicin. In particular, the present invention provides a method of treating patients suffering from a primary or metastatic liver cancer.

In the method of the subject invention, for the administration of nemorubicin, the course of therapy generally employed is from about 0.1 mg/r² to about 1000 mg/m² of body surface area. More preferably, the course of therapy employed is from about 1 mg/m² to about 1000 mg/m² of body surface area.

1. A method for treating a patient in need of a drug metabolized primarily by CYP3A, which comprises detecting CYP3A levels in said patient.

2. The method of claim 1, wherein the drug metabolized primarily by CYP3A is nemorubicin.

3. A method for optimizing the therapeutic efficacy of a drug metabolized primarily by CYP3A in a patient in need thereof, which comprises predicting the sensitivity of the patient towards said drug through the detection of CYP3A levels in a biological sample of said patient and selecting a therapeutically effective amount of said drug based on the above CYP3A levels.

4. The method of claim 3, wherein the drug metabolized primarily by CYP3A is nemorubicin.

5. A method for treating a cancer sensitive to a drug metabolized primarily by CYP3A, which comprises:
(a) obtaining a biological sample from a patient suffering from said cancer;
(b) detecting the amount of CYP3A in said sample; and
(c) selecting a therapeutically effective amount of said drug based on the above CYP3A levels.

6. The method of claim 5, wherein the drug metabolized primarily by CYP3A is nemorubicin.

7. A method for predicting patient's sensitivity to a drug, wherein said drug is metabolized by CYP3A, said method comprising determining levels of CYP3A in said patient and wherein the patient's sensitivity to said drug is effected by CYP3A activity.

8. The method of claim 7, wherein the drug metabolized by CYP3A is nemorubicin.

9. A kit for detecting the amount of CYP3A in a biological sample for use in a method for treating a cancer sensitive to a drug metabolized by CYP3A.

10. The kit of claim 9, wherein the drug metabolized by CYP3A is nemorubicin.
